(19) United States
(12) Patent Application Publication
(10) Pub. No.: US 2007/0179470 A1
(43) Pub. Date: Aug. 2, 2007

(54) Disposable transparent liposuction cannula/handle

(76) Inventor: Ella Louise Toombs, Washington, DC (US)

Correspondence Address: Ella L. Toombs, 1612 18th Street NW, Washington, DC, 20009, US

(21) Appl. No.: 11/344,483

(22) Filed: Feb. 1, 2006

(51) Int. Cl. A61M 31/00

(52) U.S. Cl. 604/500

(57) ABSTRACT

The invention is a single use, one piece, and disposable, transparent liposuction cannula/handle that gives the physician the ability to perform lipo-aspiration more safely and efficiently by immediate visualization of the aspirate at the surgical site, decreasing transmission of infectious disease, and maintaining constant vacuum power.

### FIELD OF THE INVENTION

This invention relates to the field of medical devices (cannulas) employed in the surgical removal of excess subcutaneous fat, also known as liposuction.

### BACKGROUND OF THE INVENTION

Liposuction is a cosmetic surgical procedure designed to “improve” the appearance of body contour/silhouette by aspirating (suctioning out) excess subcutaneous fat from localized areas such as: the neck, enlarged male breast, arms, abdomen, waist, flank, hips, thighs and, knees. The closed surgical system used in liposuction includes: re-usable 2-5 mm diameter×15 to 30 cm long blunt tipped, distally perforated, metal hollow tubes—referred to as cannulas. The metal cannula is screwed onto to a 12 cm black plastic hollow re-usable or permanently attached metal handle that connects to ⅝ in diameter 8 ft long clear plastic disposable tubing which empties into to a clear disposable collection canister liner which is held in a clear canister. The canister rests on and is attached to the aspirator (suctioning machine) by clear single use disposable tubing and a single use clear disposable filter. Therefore, the flow for fat extraction is through ports near the distal cannula tip, through the handle, tubing, and into the canister liner.

Currently, except for the cannula and handle (which are inserted into and/or actually touch the patient) the components of the suctioning equipment are pre-sterilized, single use and transparent—eliminating interpatient sanitization and decreasing the possibility of infection in addition to allowing the physician to visualize the aspirate. Originally, liposuction canisters were 2-liter re-usable narrow mouthed glass jars that were difficult to properly sterilize. Disposable canister liners have replaced glass jars.

The cannulas used in liposuction are manual and/or power. In manual liposuction, the physicians' dominant hand holds the black plastic (or attached metal) handle whose distal end is screwed into the re-usable metal cannula, rapidly forcing the cannula back and forth through skin incisions while suctioning underlying fibrous fatty tissue. In the case of the more recently developed “power” suctioning, a disposable powered disposable plastic handle attached to a re-usable metal cannula provides the cannula with the “power” to reciprocate or move back and forth through the tissue while suctioning with less manual force. The cannulas are in direct contact with the interior of the patients' body, in either case.

The opaque metal cannula and handle make it impossible for the physician to evaluate the quality or quantity of the aspirate without constantly looking over his or her shoulder at the clear collection tubing or at the collection canister. Ideally, as the procedure is essentially blind (12 to 30 cm of cannula in skin), the physicians' eyes should be on the patient and surgical site at all times.

Within the last 15 years, the diameter of liposuction cannulas and their distally located ports has decreased. Smaller diameter cannulas result in less tissue trauma and better healing; they are difficult to adequately sterilize between patients. Post surgery, reusable liposuction cannulas should be washed in ultrasonic washers, interior wire brush scrubbed, soaked in diluted bleach, and steam sterilization. All of which is a blind process; their opacity make it impossible to visualize residual fibrous connective tissue material that may be imbedded within the lumen. (See photograph 1, residual tissue found in cannula lumen after complete cleaning and sterilization). Those manufactured with attached metal handles (40 cm) may not fit into standard office autoclave units.

When performing manual liposuction surgery, the surgeon may use multiple cannulas requiring constant screwing on and off of the handle, resulting in increased surgical time and wear and tear on the handle/cannula interface. The torque placed on the handle/cannula attachment during liposuction causes breaks in the metal to plastic “bond” further increasing surgical time. Longer surgical times are associated with increased adverse events. Liposuction is a cosmetic procedure and occurrences of adverse events should be minimal to absent.

Hazards associated with liposuction can include bleeding, organ perforation and transmission of infection disease. In the case of hepatitis C or HIV, infection could occur and go undiagnosed for many years.

In summary, the currently available liposuction cannulas/handles are difficult to sanitize, do not give the physician the ability to immediately evaluate the aspirate, and are inefficient.

### BRIEF SUMMARY OF THE INVENTION

The problems described in the foregoing paragraphs would be avoided with clear, disposable liposuction cannulas/handles designed to give the surgeon the ability to visualize the material being suctioned and maintain the sterility of the procedure by being disposable as is the remainder of the equipment used in liposuction. Single patient use eliminates sterilization problems.

In the event of organ perforation, bleeding, or problems with fat aspiration the transparency of the invention would allow the surgeon to respond immediately. As the handle is attached, the need for screwing on and off or replacement during the procedure is avoided.

The invention is a transparent, single patient use liposuction cannula/handle. The claims for the invention are that it will: 1) allow the surgeon to immediately visualize the liposuction aspirate increasing safety and efficiency and 2) be a single use disposable tool minimizing the transmission of infectious disease.

### DESCRIPTION OF THE DRAWINGS

FIG. 1. is a side view of the cannula demonstrating the lateral ports proximal to the distal (patient) blunt tip opening into the lumen—illustrated by the continuous straight line spanning the cannula length? The proximal end (physician) of the cannula body is molded for attachment to a power handle.

FIG. 2. is a bottom view of the cannula body/handle demonstrating: FIG. 2¹ recessed physicians thumb placement in the handle cannula, FIG. 2² lateral and central ports in continuity with the lumen of the cannula body/handle. (Semi-circular hatching infer transparency of the cannula and handle)

FIG. 3. is a cross-section of the cannula wall and lumen

FIG. 4. is an enlarged section of FIG. 2³

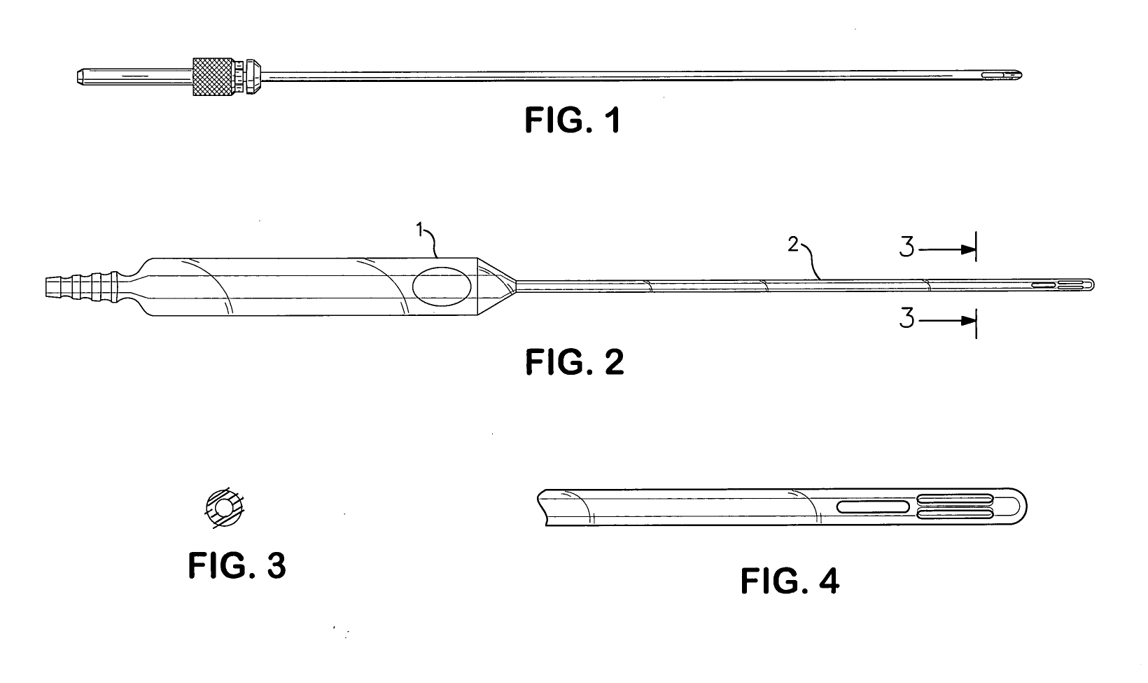

### DETAILED DESCRIPTION OF THE INVENTION

The invention is a completely transparent, one-piece liposuction cannula/handle designed to increase the safety and effectiveness of liposuction by virtue of being transparent and disposable. While the present invention has been described is its basic form, it is understood that description is provided by way of example and that numerous changes may be made without departing from the spirit and scope of the invention. Due to the amount of tissue force required in liposuction and the narrow diameter to shaft length ratio, the cannula and handle will be injection molded from a medical grade polycarbonate mimicking the strength of stainless steel and suitable for ethylene oxide or gamma radiation sterilization. Manufacturing will occur in a facility adhering to the CFR's and FDA regulations.

References:

U.S. Patent Documents

| | | | |
|---|---|---|---|
| 4,863,439 | September 1989 | Sanderson | 604/264 |
| 5,052,999 | October 1991 | Klein | 604/22 |
| 5,181,907 | January 1993 | Becker | 604/22 |
| 5,472,416 | December 1995 | Blugerman | 604/35 |
| 5,817,050 | October 1998 | Klein | 604/35 |
| 6,113,569 | September 2000 | Becker | 604/35 |
| 6,638,238 | October 2003 | Weber | 604/22 |

Note:
all references are for metal liposuction cannulas - generally

What is claimed is:

1. transparent liposuction cannula/handle unit designed for single patient use.
